(19) United States
(12) Patent Application Publication
(10) Pub. No.: US 2006/0241428 A1
(43) Pub. Date: Oct. 26, 2006

(54) Method of Generating an Ultrasonic Image

(76) Inventor: Chih-Hung Kao, Taipei Hsien (TW)

Correspondence Address: NORTH AMERICA INTELLECTUAL PROPERTY CORPORATION, P.O. BOX 506, MERRIFIELD, VA, 22116, US

(21) Appl. No.: 10/908,235

(22) Filed: May 3, 2005

(30) Foreign Application Priority Data
Mar. 11, 2005 (TW) 094107512

(51) Int. Cl. A61B 8/00

(52) U.S. Cl. 600/437

(57) ABSTRACT

Generation of an ultrasonic image includes producing an ultrasonic image, selecting a predetermined area on the ultrasonic image, and changing a color of the predetermined area. This will allow a user to indicate a predetermined area according to individual needs and to color a predetermined area. Thus the kernel portion of the ultrasonic image can be emphasized.

### BACKGROUND OF INVENTION

1. Field of the Invention

The present invention relates to a method of generating an ultrasonic image, more particularly, to a method of generating an ultrasonic image with a predetermined edited area.

2. Description of the Prior Art

In the fast development of medical equipment, diagnostic ultrasound is utilized for detecting internal organs and the blood flow of the human body. The speed of sound is constant in a predetermined medium and frequency is relative to wavelength. When the sound wave transmits through the medium, its intensity gradually weakens. The primary cause for this weakening is due to absorption. When the absorption of the frequency and the sound wave form a linear relation, as the frequency increases, the energy weakens. The proximity of impedance and medium of the sound wave is related to the speed of sound; this is a basic characteristic of matter. When the sound wave transmits, different matter has different impedance and the interface will generate reflection and refraction. For most human body construction, when the sound passes through the interface most of the energy passes through, however, when the sound encounters the lung (i.e., air) and skeleton, as it passes, it is unable to diagnose as it is mostly reflected.

A transducer called a probe is the source of an ultrasonic scanner. There are two methods of generating sound waveforms, these are: continuous waves and pulse waves. The former method, continuous waves, utilizes Doppler ultrasonic for detecting blood flow. The latter, pulse waves, utilizes images formed. Echo signals reflected by the human body construction are utilized to display the ultrasonic image electronically on a monitor. As the instantaneous ultrasound is a type of dynamic image, when utilized, inspection is easy. Ultrasound is simple to operate, it does not rely on the operator, hence inspection time and expense become more economical, it will not endanger the life of the patient and there is no exposure to radioactive rays.

The ultrasonic image in the market place utilizes the principle of sound wave reflection against objects to form an image. The sound reflection can only analyze the shape of the object. It is unable to distinguish the object's color, therefore, it can only be outputted to a black and white heat sensing printing device. For a common colored ultrasonic wave, the image is transferred from black and white to a brown tone, a blue color or a red color that represent different directions of the blood flow, however, this method is unable to achieve the effect of full color. As medical help is becoming more common, the patient strives for the best consultation by seeking opinions from different doctors regarding the illness. Each doctor will provide a subjective opinion by studying the black and white ultrasonic image. One doctor's subjective opinion may neglect the previous doctor's cognition; this may caused different interpretations of the same ultrasonic image. In addition, when the patient and family members see the black and white ultrasonic image, it is most likely to cause a strange and frightening psychological effect for them. If, during the diagnosis, each doctor can emphasize a suspicious area in the ultrasonic image then other doctors can take note of the suspicious area during subsequent diagnosis or even in the future after treatment. Through the comparison of the suspicious area in the ultrasonic image before and after treatment, the results of the treatment can be better understood. As for the doctor, the patient and the family members, such ultrasonic wave image will help to increase the quality of medical treatment as well as the treatment relationship between the doctor and the patient and family members.

### SUMMARY OF INVENTION

Therefore the main objective of the claimed invention is to provide a method of generating an ultrasonic image to solve the above problems.

The claimed invention provides a method of generating an ultrasonic image, the method comprising producing an ultrasonic image, selecting a predetermined area on the ultrasonic image, and changing a color of the predetermined area.

These and other objectives of the present invention will no doubt become obvious to those of ordinary skill in the art after reading the following detailed description of the preferred embodiment that is illustrated in the various figures and drawings.

### BRIEF DESCRIPTION OF DRAWINGS

FIG. 1 illustrates a diagram of ultrasonic display system according to the present invention.

FIG. 2 illustrates a display on a user interface of a second monitor after an image processing software is activated.

FIG. 3 illustrates a diagram of an ultrasonic image of the present invention.

FIG. 4 illustrates a diagram of a picture frame and a text space provided by an image processing software of the present invention.

FIG. 5 illustrates a diagram of an ultrasonic image of an image processing software after printing via a printing device.

### DETAILED DESCRIPTION

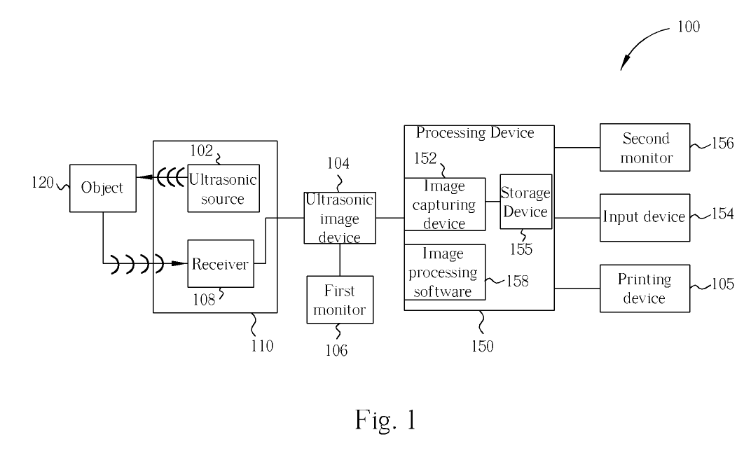

Please refer FIG. 1. FIG. 1 illustrates a diagram of an ultrasonic display system 100 according to the present invention. An ultrasonic source 102 within a probe 110 of the ultrasonic display system 100 will transmit an ultrasonic signal to an object 120. The transmitted ultrasonic signal will be reflected against the object 120 and received by a receiver 108. The receiver 108 will convert the ultrasonic signal into an electrical signal. The electrical signal will be transmitted to an ultrasonic image device 104 according to a difference in intensity of the reflected ultrasonic signal. The ultrasonic image device 104 then generates an ultrasonic image according to the electrical signal. The user is able to observe the ultrasonic image generated by the ultrasonic image device 104 via a first monitor 106. The image generated by the ultrasonic image device 104 will be captured by an image capture device 152 within a processing device 150. The user can observe the captured image via the second monitor 156. At this time, the images displayed on the first monitor 106 and the second monitor 156 are synchronized. The ultrasonic image captured by the image capturing device 152 will be stored in a storage device 155 (e.g., a hard disk).

The traditional ultrasonic image is mostly black and white. This is true even though dynamic colored images can be generated by the Doppler image processing method. One common utilization for dynamic colored Doppler image processing is in the utilization to determine internal blood flow. However, the area of the internal blood flow is displayed in a brown color or other colors while the rest of the active organs continue to be displayed in black and white. As a result, this particular image is not obvious. In order to allow the user to observe characteristics of the image quickly and accurately, in this embodiment, the user further utilizes an input device 154 (e.g., a keyboard, a mouse or a touch pen) to operate an image processing software 158 stored in the processing device 150 to specially process the image captured in the image capturing device 152. The processed image will then be displayed on the second monitor 156. Finally, a printing device 105 is capable of printing the results displayed on the second monitor 156.

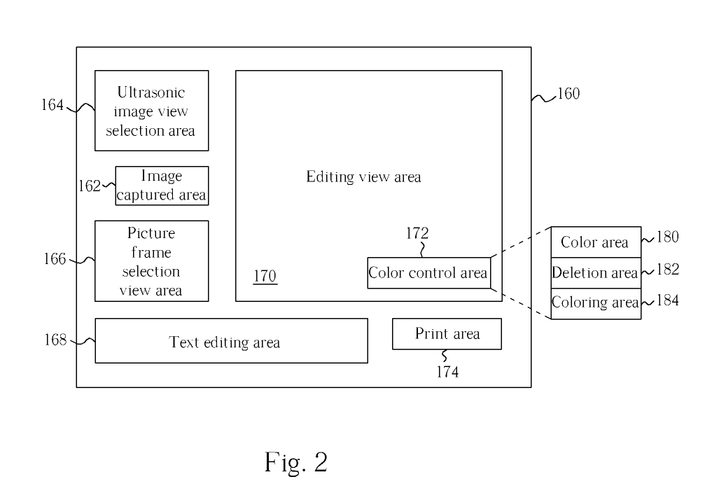

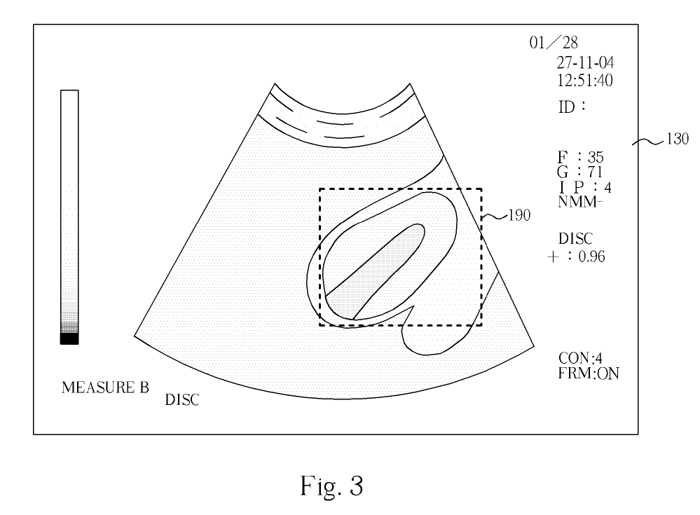

Please refer to FIG. 1, FIG. 2 and FIG. 3 all at the same time. FIG. 2 illustrates a display on a user interface 160 of a second monitor 156 after an image processing software is activated. FIG. 3 illustrates a diagram of an ultrasonic image of the present invention. After the user executes the image processing software 158, the user interface 160 will be activated. The user interface 160 includes an ultrasonic image view selection area 164, image captured area 162, picture frame selection view area 166, text editing area 168, editing view area 170, color control area 172, print area 174 and so on. The user is able to observe a plurality of the ultrasonic images stored in the storage device 155. This plurality of ultrasonic images having been obtained by the ultrasonic image device 104, in the processing device 150 and viewable via the ultrasonic image view selection area 164. An ultrasonic image 130 can be captured after the image captured area 162 is activated and the selected ultrasonic image is then displayed at the editing view area 170. The color control area 172 can be directly executed on the editing view area 170. At this time, the color control area 172 will display at least three function areas: color area 180, deletion area 182, and coloring area 184. When the user selects the color area 180, the user can decide the color such as red or blue to be applied onto the ultrasonic image of the editing view area 170. The user then utilizes the input device 154 to move and navigate the ultrasonic image of the editing view area 170 in order to color a predetermined area 190. If the user is not satisfied with the result of the colored predetermined area 190, the user can execute the deletion area 182 to delete the entire coloring result. Once the coloring action is completed, the coloring area 184 is executed, and the ultrasonic image of the editing view area will display the result of the coloring. Later, the user is also able to select the color area 180 to change the color of the ultrasonic images in the editing view area 170.

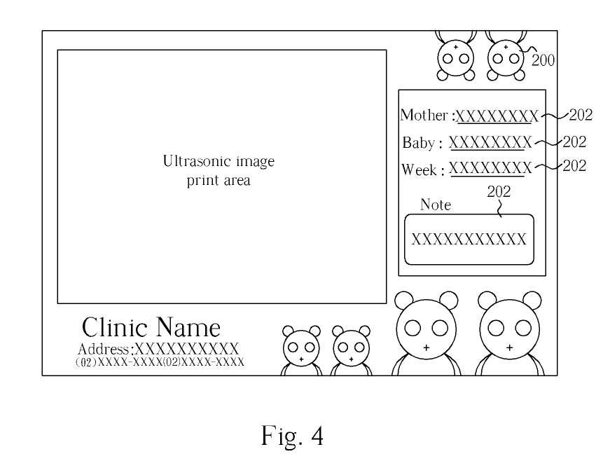

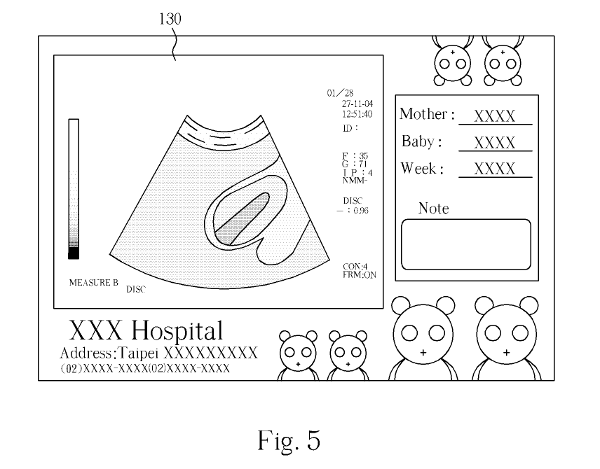

In addition to the function of coloring the ultrasonic image in the above-mentioned, the image processing software 158 of this embodiment provides another function. Please refer to FIG. 4 and FIG. 5. FIG. 4 illustrates a diagram of a picture frame and a text space provided by the image processing software 158 of the present invention. FIG. 5 illustrates a diagram of an ultrasonic image of an image processing software after printing via a printing device. When the user selects the picture frame selection view area 166, there will be a plurality of template picture frames for the user to choose from, as shown in a picture frame 200 with a plurality of text spaces 202 of FIG. 4. The user is able to edit the contents of the plurality of text spaces 202 via the text editing area 168. The results of the edits can be viewed in the editing view area 170. Finally, the user can utilize the printing device 105 to print the images that are displayed on the second monitor 156.

Please note, the first monitor 106 and the second monitor 156 of FIG. 1 can be synchronized to display the image detected by the ultrasonic image device 104, or the first monitor 106 and the second monitor 156 can be configured to be a single monitor.

As the ultrasonic image is the echo signal of the human construction displayed on the monitor electronically, it is divided into three kinds of modes, A mode, M mode and B mode: The A mode displays the vertical peak of the echo received, the horizontal axis represents time or distance, the vertical axis represents amplitude or intensity of the echo. The M mode is time motion scan, for tracing the relationship of the pattern of the motion of the object structure and time, the vertical axis represents the depth of the reflected wave, the horizontal axis represents the time, commonly utilized in ultrasonic figures. The B mode is luminance adjustment, the echo signal is indicted by luminescent spot, which records the signal of each scan to display an image of extreme contrast by a bistable pattern method, and the image is displayed in gray tone. The fine image can distinguish the intensity of the echo signal. The method of the present invention is suitable for utilizing any of the three kinds of ultrasonic image modes.

The conventional outputted ultrasonic image is utilized based on the principle of reflection from an object to form an image, which collocates with a black and white heat sensor printing device to produce the output. Only a black and white result can be outputted utilizing the conventional ultrasonic image. The present invention provides a method capable of coloring a selected predetermined area on a black and white ultrasonic image, and to combine a template picture frame to produce an entire color output. Besides the image captured from the ultrasound, a text signal can also be outputted, such as patient medical service information, date, doctor diagnosis and so on. This additional information helps to ensure that the output content of the ultrasonic image output is clear and detailed. In order to have a clearer output of the ultrasonic image, a special color is utilized to mark the important area to improve richness of the ultrasonic image output, and to allow the patient and the family members to have a clearer understanding of the meaning of the ultrasonic image.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of generating an ultrasonic image comprising:
producing an ultrasonic image;
selecting a predetermined area on the ultrasonic image; and
changing a color of the predetermined area.

2. The method of claim 1 further comprising combining the ultrasonic image with a predetermined image.

3. The method of claim 2, wherein the predetermined image comprises a picture frame and a text object.

4. The method of claim 2 further comprising printing the ultrasonic image with the predetermined image.

5. The method of claim 1 further comprising printing the ultrasonic image combined with the predetermined area after changing the color of the predetermined area.

6. The method of claim 1 wherein the ultrasonic image complies with a format of an A mode, a B mode or an M mode.
